(19) United States
(12) Patent Application Publication
(10) Pub. No.: US 2004/0209373 A1
(43) Pub. Date: Oct. 21, 2004

(54) Method and apparatus for the determination of water in materials

(75) Inventor: Theodore B. Lynn, Hamden, CT (US)

Correspondence Address: Stephen R. Finch, Wolf, Greenfield & Sacks, P.C., 600 Atlantic Avenue, Boston, MA, 02210, US

(73) Assignee: DEXSIL CORPORATION, HAMDEN, CT

(21) Appl. No.: 10/847,553

(22) Filed: May 17, 2004

Related U.S. Application Data
(63) Continuation of application No. PCT/US01/44152, filed on Nov. 26, 2001.

(51) Int. Cl. G01N 33/18

(52) U.S. Cl. 436/39

(57) ABSTRACT

A method and apparatus for determining water content in materials. A method and apparatus are provided whereby water contained in a sample of uncured cement is extracted into a solvent. Some of the solvent may then be reacted in a vessel and an indicator, such as a gas may be produced in a quantity that is proportionate to the concentration of water contained in the cement composition. By measuring the amount of indicator produced, the amount of water in the original sample can be determined.

### RELATED APPLICATIONS

This application is a continuation of International Application No. PCT/US01/44152 filed Nov. 26, 2001, which was published under PCT Article 21(2) in English and is incorporated by reference herein.

### FIELD OF THE INVENTION

The present invention is directed to a method and apparatus for determination of water content in materials, and particularly, to the determination of the water content in solids and slurries that are difficult to extract.

### BACKGROUND OF THE INVENTION

The structural integrity of materials made from cement, concrete and similar materials is often a function of the composition of the material being used. For example, the amount of water that is added to cement based materials in the mixing process may have a direct affect on the quality of the final cured product. For instance, uncured cement products such as concrete typically having a water content of between 8 and 15% may be considered adequate for many uses. However, water content greater than about 15% or less than about 8% may start to degrade the properties of the final cement or concrete product. Multiple guidelines and regulations exist providing upper and lower limits on the amount of water that is to be used to make concrete to be used in different applications. However, these ranges are frequently not adhered to, resulting in weak or unsafe structures.

In order to check the water content of an uncured cement sample, several tests have been developed. One of these tests, referred to as the slump test (see ASTM C-143), involves measuring the time it takes for a cone shaped sample of uncured concrete to collapse. This test often provides an adequate estimate as to the water content in the sample when the test is run properly. However, results may vary with different operators, and results may be skewed by additives that have become available and may be placed in cement and concrete products for the purpose of, for example, improving viscosity, pourability, etc. Thus, if a sample contains one or more of the additives, it may result in an end product that is out of specification while a properly run slump test may indicate that the sample is acceptable.

As moisture content may play an important role in any number of different matrices, different tests have been developed for measuring moisture in, for example, oil, solvents and some solid matrices, such as soil. These methods include, for example, gravimetric analysis, Karl Fischer titration, conductivity testing and neutron activation analysis. One of the tests that may be used to measure the moisture content in soil is the HYDROSCOUT® moisture test system (available from Dexsil Corporation, Hamden, Conn.) that includes removing water from soil with a compatible solvent, reacting the solvent with a reagent to produce a gas, and quantitatively measuring the amount of gas produced. This test provides accurate, reliable results for most soil samples and may be useful, for example, in agricultural applications and in agricultural planning. Moisture in soil may also be important to measure when performing compaction studies. Some forms of earth such as clay are difficult to extract and thus do not provide results as favorable as those provided by other soil types. It is believed that these clays exhibit a molecular structure that binds any water strongly enough to inhibit the ability of a solvent to pull the water away from the clay's molecular structure. Extended extraction time periods may provide better results when these clays are to be analyzed, but extraction ratios are less than 50% and highly variable.

### SUMMARY OF THE INVENTION

In one aspect, the invention provides for a method of determining water content in cement comprising the steps of providing a sample of plastic cement, mixing a solvent with the sample to produce an extract and adding a reagent to the extract to produce an indicator.

In another aspect, the invention provides a method for assessing the quality of concrete comprising extracting water from a plastic concrete sample into a solvent, adding a reagent to the solvent, producing a gas in an amount proportional to the amount of water in the solvent, determining the amount of gas produced and assessing the quality of the concrete sample.

In another aspect, the invention provides a method comprising extracting water from a cement sample, reacting the water with a reagent to produce a gas and determining the amount of gas produced.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, in some figures not every component is labeled, nor may every component of each embodiment of the invention be shown where illustration is not necessary to allow those of ordinary skill in the art to understand the invention.

### BRIEF DESCRIPTION OF THE DRAWING

FIG. 1 provides a cutaway view of a test instrument and test compartment including reagents;

FIG. 2 provides a graph that illustrates a regression analysis for results achieved practicing an embodiment of the invention; and

FIG. 3 provides a graph that illustrates a regression analysis for additional results achieved practicing an embodiment of the invention.

### DETAILED DESCRIPTION

The present invention is directed to a method and apparatus for the determination of water content in solid matrices. The method can provide accurate results in the percentage or ppm range for water content of difficult to extract solids and slurries, such as cement and concrete.

As used herein, the term “cement” is used as it is understood by those skilled in the art, i.e., a pulverized mixture of compounds used to produce materials such as mortar and concrete. When mixed with water and allowed to set, it will harden into a structural material. Typically, cement may be produced by combining various proportions of alumina, silica, lime, iron oxide and magnesium oxide and burning them together. “Concrete” is defined herein as a mixture of cement and an aggregate, such as sand or gravel and is typically used in the construction of roads, bridges, buildings, etc. As used herein, cement or concrete is “plastic” after it has been mixed with water and prior to its cementation into a hardened material. “Extract,” as used herein, refers to a solvent or solvent mixture after it has been commingled with a sample in order to remove at least a portion of any water contained in the sample. An “indicator,” as used herein, refers to a change of state, form, temperature or appearance, such as may occur with a chemical, physical or electrical change, and shows an operator that water, or a particular amount of water, is present in a sample being tested. Some examples of indicators are a change in temperature, a change in color, a change in pressure or a change in conductivity.

In one embodiment, a sample containing plastic cement is extracted with a solvent to remove some or all of the water from the cement sample. The extract is reacted with a reagent and any water contained in the extract reacts with the reagent to form a gas. The amount of gas produced is proportional to the amount of water in the extract and thus can provide an indication of the amount of water contained in the extract. Once the water content of the extract is known, the water content of the cement sample can be accurately calculated if the water can be consistently extracted and reacted.

Although the process disclosed herein may be useful in a variety of matrices such as soil, foodstuffs and biological materials, it may also be useful with solids or slurries that include species that react with water. Water in some matrices, such as sand, soil and gravel may be easily extracted by a number of polar solvents, however, other matrices, such as some clay samples, are difficult or impossible to extract efficiently due to the chemical entrainment of water in the matrix. Also difficult to extract are samples containing cement, which not only adsorbs water but starts to chemically react with it soon after mixing. Thus, it is believed that the amount of water extractable from a cement or concrete sample should depend upon how long the sample has been reacting and that only remaining free, or unbound, water could actually be separated from the sample. The present method, however, can efficiently extract water from cement or concrete materials at any point during which the sample is plastic.

In one embodiment, the testing procedure is started by taking a representative sample of the material to be tested. Material may be measured by volume or by weight and homogeneous matrices can be sampled directly and may result in an accurate reading without further calculation. For non-homogeneous samples, for instance, concrete samples that include different sized pieces of gravel, the operator can opt for measuring the moisture content of the entire sample, or may first separate the gravel or other non-homogeneous pieces from the sample to produce a homogeneous, or nearly homogeneous, sample. If aggregate is not removed from the sample, precise results may require large samples to assure statistically accurate readings, and these large samples may be particularly difficult to analyze. If gravel is first removed from a concrete sample, for instance, by sieving, a smaller sample can be used to arrive at a useful value for the slurry component of the concrete. In this manner, the operator can be assured of more consistent, more representative results.

In another embodiment, a solvent may be added to the sample in order to extract at least a portion of the water that may be contained in the sample. The solvent may extract, for example, more than 70, 80 or 90% of the water from the sample. Preferably, the solvent is capable of solubilizing water and is non-reactive with a reagent that may be used to determine the amount of water contained in the solvent. More preferably, the solvent is capable of quickly and efficiently removing water from the matrix with a minimal amount of agitation, and it is also preferred that the solvent allows the matrix to settle out after agitation, to allow the transfer of solvent containing a minimal amount of suspended solids. In another embodiment, the solvent is added to the matrix being analyzed in a ratio that provides for an efficient extraction of water from the matrix while minimizing the total volume of solvent required. For example, the ratio of solvent to solid sample may be 1 to 1, 0.5 to 1, 2 to 1 or 3 to 1 on a wt/wt basis. More preferably the solvent is a polar solvent or has polar characteristics. For example, the solvent may be an alcohol, ester, aldehyde or ketone. Mixtures of polar and less polar solvents may also be used. For example, 100% ethanol or methanol or a 20/80% mixture, by volume, of methanol/THF may be used.

The solvent and sample may be comingled, such as by agitating. The solvent and sample may be agitated in any number of ways including for example, shaking, mixing and sonicating. The mixture may also be heated to promote extraction. Preferably, the sample and solvent are shaken for a period of less than one or two minutes while extracting up to or greater than 90, 95 or 99% of the water contained in the sample. In a preferred embodiment, there is no need to add additional agitators, such as steel balls, in order to break up the matrix and assure mixing of the two phases. The choice of an appropriate solvent and mixing vessel can provide for adequate extraction from a variety of cement and concrete samples. In another embodiment, the solvent and sample are allowed to separate after agitating and in order to observe this separation, the vessel in which the agitation takes place may be transparent or translucent so that the operator can evaluate the amount of settling that has taken place. Although under some conditions, the reactions to follow may be performed on a mixture of the sample and extract, it is often preferred that the extract be separated from the extracted sample. The extract can simply be decanted off of the top of the sample or it can be removed with, for example, a pipette or syringe. The extract may also be filtered to remove any suspended material that may be undesirable in the solvent.

In another embodiment, the extract containing at least a portion of any water originally contained in the sample being tested, may be reacted with a reagent to produce a gas such as hydrogen or acetylene. In order to contain the gas produced, it may be preferred to perform the reaction in a closed compartment. In further efforts to contain any gas that is produced during the reaction, it may be preferred to introduce the reagent to the solvent without opening the compartment. This may avoid the loss of some of the gas that may be formed during the initial reaction between the reagent and any water contained in the solvent. One method of doing this may be to perform the reaction in a compartment that is flexible, such as a polyethylene screw-top tube. Preferably, at least a portion of the compartment is flexible. The flexible compartment may contain a secondary container enclosing a reagent that is isolated from the interior of the vessel by the secondary container. The reagent may also be isolated from any solvent the flexible compartment may contain, but can be introduced to the solvent when mixing is desired. In one embodiment, the secondary container may be a smaller, frangible container, such as a glass ampule. Thus, by compressing the sides of the flexible compartment, the glass ampule can be broken within the vessel, allowing the reagent to mix and contact any solvent contained therein. It is therefore preferred that the compartment be inwardly flexible, allowing the breakage of an ampule contained therein. It is also preferred that the compartment exhibit a fixed maximum volume in order to minimize or eliminate expansion of the compartment upon pressurization when gas is produced inside the vessel. The compartment may be made of a polymer of sufficient puncture resistance to resist piercing by glass fragments. HDPE of about 30-40 mil. thickness can be used.

The reagent may be any compound or chemical that will provide an indication when reacted with water. Preferably the compound reacts only with water and not with other compounds containing hydroxyl groups such as alcohols, aldehydes and ketones. It is preferred that the reagent form a gas when reacted with water and may be selected from alkaline metals, alkaline metal hydrides and alkaline metal carbides. More preferably, the reagent is selected from sodium, calcium hydride and calcium carbide and most preferably is calcium hydride. The reagent may be suspended or dissolved in a carrier and it is preferred that the carrier be inert to the reagent. The carrier may be chosen based on a variety of parameters, such as the ability to disperse the reagent as quickly and completely as possible in the extract. In addition, the carrier may be chosen to facilitate production and storage of the reagent. For example, if calcium hydride is used, it may be suspended in a non-reactive carrier such as kerosene or transformer oil.

The amount of reagent used should be enough to react with all, or substantially all, of any water that may be in the extract solvent. The amount of reagent may also be limited so that if an excessive amount of water is present, the resulting pressure build-up does not result in the failure of the compartment.

After reacting any water contained in the extract with a reagent to produce a gas, the amount of gas formed can be measured in a number of ways including change in volume and change in pressure. A variety of techniques are available for determining the resulting pressure in the compartment including, for example, directly measuring the pressure inside the compartment or measuring the rigidity or resistance to compression that a flexible compartment may exhibit. In one embodiment, the pressure may be measured by introducing a probe into the interior of the compartment while minimizing any leakage that may occur during the procedure. A portion of the compartment may be designed so that it is susceptible to piercing by a probe, such as a hollow pressure pin. The compartment may be open at one end and may include a threaded area for receiving a screw cap. The screw cap may be formed from a rigid material such as polypropylene so that it may be securely tightened to the open end of the compartment. The cap may further include an orifice into which a septum can be positioned. For example, the septum may be secured between the rim of the compartment and an interior collar or other surface of the cap. In this manner, the compartment may be securely sealed to prevent the loss of reagents or pressure yet may be readily available for sampling of pressure via a pressure pin or other probe. The probe may be in fluid communication with a pressure detection device so that the device is exposed to whatever pressure the lumen of the pressure pin is exposed to. For example, the probe may be in fluid communication with a pressure transducer that can produce an electrical signal in response to a change in pressure. Preferably, the probe is securely fastened to the pressure transducer such that when the probe enters the pressure vessel, the interior pressure in the vessel is communicated immediately and directly to the pressure transducer. The probe and pressure transducer may be configured to minimize the interior volume of each, so that the pressure inside the compartment is only slightly reduced by the resulting gas expansion into the probe when the probe enters the compartment. The probe may be surrounded by an outer sheath, or housing, designed to receive the pressure vessel, the sheath providing protection and guidance for piercing the septum with the probe. Preferably, the septum, or another portion of the pressure vessel, is made of material that can form a pressure tight seal with the probe and is also inert to any solvents or reactants that are used in the test. For example, a silicone rubber septum has been found to provide adequate results.

After the pressure in the interior of the compartment is transmitted to the pressure detector, more useful information may be obtained by converting that pressure to a quantity or concentration of water. With a compartment of fixed volume, the ideal gas law may be used to convert a pressure reading to an amount of gas produced and the reaction stoichiometry can be used to convert the amount of gas into a water equivalent. If the size of the sample extracted and the amount of extract reacted are also known, the concentration of water in the original sample can also be calculated. If the extraction efficiency of the method is less than 100%, this reduced extraction efficiency may also be taken into account and compensated for in performing any calculations. Preferably, the extraction efficiency is greater than 95 or 99% so that a variety of samples can be run with a single program. Corrective calculations may be performed manually or by a microprocessor that is in communication with the pressure sensor. The microprocessor may be programmed to provide a readout in various units, such as mg, ppm or percentage water, or may provide a simple go, no go result such as providing a red light or a green light depending on the acceptability of the concrete material being tested based on its water content. Those skilled in the art will recognize that results can be reported in any number of ways such as in ppm on a mass/mass basis, mass/volume basis, volume/volume basis or in a percentage, ratio or fractional reading.

To provide for the safe disposal of the pressure vessel and associated testing equipment, a small amount of water or other reactive material may be added to any excess reagent that might remain in the tube after completion of the reaction. This may destroy any leftover reagent, resulting in a relatively innocuous material. Such water or other reactive material may be held in another secondary container, such as a crushable glass ampule.

### EXAMPLES

The following examples provide data produced by employing various embodiments of the invention that illustrate, among other features, the accuracy, precision, level of detection, repeatability, and the broad applicability of the method with various cement products at different stages of the cementation process. In addition, data are provided that show the inapplicablity of the method to kaolinite clay, a material that binds water, much as cement does.

### Example 1

Concrete samples were analyzed for water content using one embodiment of the invention to determine appropriate minimum detection limits (MDL) for the technique. Concrete samples that had been previously oven-dried at 110° C. were first analyzed prior to adding water, so that a blank reading could be determined. Samples were then mixed to contain 2% water. To determine MDLs, it is recommended that levels of analyte be no more than 10 times the expected MDL (40 CFR Part 136 Appendix B Rev 1.1.1). Thus, although 2% is below the expected water concentration for a normal plastic concrete sample, this level was used to supply important information about the capabilities of the method.

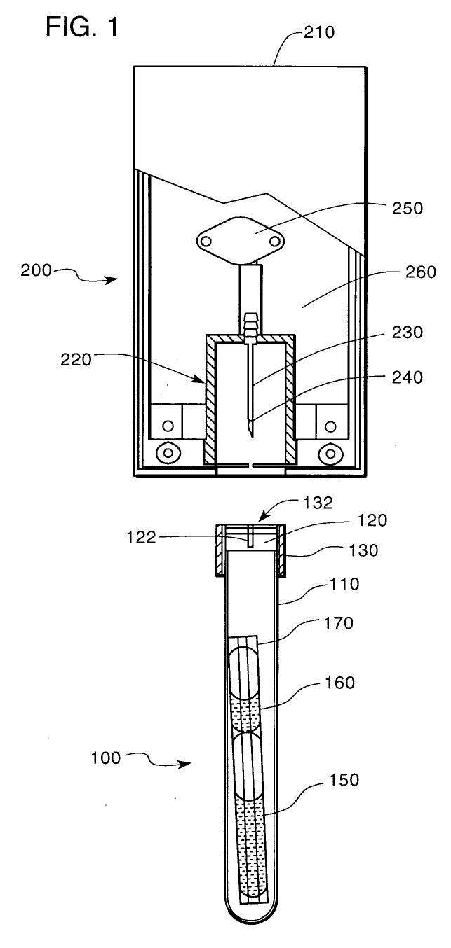

A 10 g sample of oven-dried cement taken from the concrete, excluding any aggregate (2.36 mm mesh screened), was placed in a 50 mL polypropylene centrifuge-type extraction tube, and weighed. The sample was then spiked with 0.2 g water, mixed, and allowed to stand for 15-30 minutes. The contents of a glass, snap-top vial containing absolute ethanol were added to the extraction tube containing the sample after breaking the snap-top off of the vial. After 10 mls of the extraction solvent were added to the extraction tube, a screw cap was placed tightly on the tube and its contents were then manually shaken for a period of about 1 minute. During this time, it was seen that the concrete sample became dispersed and intermixed with the solvent. At the completion of the period of shaking, the sample was allowed to settle for about 2 minutes during which time the solvent rose to the top and the sample settled to the bottom. A polypropylene syringe having a fixed volume of 0.25 mL was then used to draw a sample from the extraction tube. The full contents of the syringe were then dispensed into a polyethylene reaction tube 110 (FIG. 1). A pressure fit stopper 120 (septum) was then placed into the top of the tube by firmly seating it into the opening. A white screw cap 130 with a circular orifice 132 in the middle was then tightened onto the threads of the tube forming a seal between the cap 130, stopper 120 and the interior surface of the opening of the tube.

A calcium hydride suspension was produced by suspending 84 mg of calcium hydride in 0.5 mL mineral oil to produce a 20% suspension. This suspension was then diluted to improve flow by adding 1.0 mL of “high flash” aromatic solvent, a commerically-available aromatic solvent having a flash point of greater than 150° F. (>65.6° C.). A frangible glass ampule 150 containing calcium hydride and a disposal ampule (water) 160 were both supported in tube 110 by a compressible polypropylene holder 170. The calcium hydride ampule 150, containing 420 milligrams of calcium hydride suspension in 1.0 mL of aromatic solvent, was broken by squeezing the sides of the polyethylene reaction tube in the vicinity of the glass ampule. The breakage of the glass ampule introduced the calcium hydride to the extract. The extract and the calcium hydride were then mixed together by shaking the tube vigorously for about 30 seconds. The tube was allowed to stand for about 1.5 minutes and then shaken again for about another 30 second period. The mixture was then allowed to stand for an additional 30 seconds. A pressure meter 200 (HYDROSCOUT® test meter, Dexsil Corporation, Hamden, Conn.) was set for “program D” and the display on the LCD readout 210 of the meter read “tube.” The capped end of the tube was then firmly fitted into the sleeve 220 of the meter 200 allowing pressure pin 230 to puncture the septum at guide 122 that passes a portion of the distance through stopper 120. The pressure pin 230 was then allowed to communicate with the interior of the reaction tube via the pressure port 240 and the lumen of the pressure pin. Pressure pin 230 also communicated with pressure transducer 250 that in turn provided a signal to printed circuit board 260. The “read” button on the meter 200 was pressed and after displaying the word “calc” for several seconds, the results were displayed in milligrams of water. To determine the weight percent of the water in the concrete, the reading in milligrams was divided by ten times the weight (in grams) of the soil. Minimum detection limit (MDL) results are provided below in Table 1. Blank results are provided in Table 2, the average of which were subtracted from the detected readings to result in the calculated values provided in the last column in Table 1.

The results provide an average reading of 1.989% water (blank subtracted) on samples that were prepared to contain 2.0% water. The calculated MDL of 0.365% water is well below the anticipated practical testing range of 10-20% water and demonstrates the low level applicability of this test method.

TABLE 1
Minimum Detection Limit Determination

| | | Wt. Water | | | Result less |
|---|---|---|---|---|---|
| Sample ID | Wt. (g) | (g) | Result (mg) | Calc % | Blank (%) |
| 1 | 10.056 | 0.195700 | 206 | 2.05 | 1.842 |
| 2 | 10.009 | 0.197200 | 211 | 2.11 | 1.901 |
| 3 | 10.029 | 0.198700 | 240 | 2.39 | 2.186 |
| 4 | 10.039 | 0.199100 | 223 | 2.22 | 2.015 |
| 5 | 10.009 | 0.197100 | 229 | 2.29 | 2.081 |
| 6 | 10.033 | 0.198000 | 215 | 2.14 | 1.936 |
| 7 | 10.001 | 0.197900 | 217 | 2.17 | 1.963 |
| Average | 10.025 | 0.197671 | | | 1.989 |
| Std Dev | | 0.001132 | | | |
| | | | Std Dev | 0.116099 | |
| | | | MDL | 0.364552 | |
| | | | 95% C.I. +/− | 0.10738 | |
| | | | Theoretical | 1.972 | |
| | | | Result (%) | | |

TABLE 2
Blank Determination

| | Sample | Result | Weight Percent |
|---|---|---|---|
| Sample ID | Wt. (g) | (mg) | Water Detected |
| Blank 1 | 10.0342 | 25 | 0.249148 |
| Blank 2 | 10.0037 | 23 | 0.229915 |
| Blank 3 | 10.0073 | 22 | 0.219840 |
| Blank 4 | 10.0508 | 26 | 0.258686 |
| Blank 5 | 10.0171 | 21 | 0.209642 |
| Blank 6 | 10.0011 | 20 | 0.199978 |
| Blank 7 | 10.0000 | 8 | 0.080000 |
| | | Mean | 0.206744 |

### Example 2

In order to check the usefulness of the method described in Example 1 on a matrix known to tightly bind water, the same method and apparatus as used in Example 1 were used to test for water in kaolinite clay. A batch of kaolinite clay was prepared, after oven drying, to contain 28.7% water by weight. The clay was split into multiple samples and individual samples of approximately 5 grams (actual weights recorded) were tested using the procedure described in Example 1. Results are shown below in Table 3 and indicate a poor and variable water extraction efficiency of less than 50%. This indicates that this method cannot provide useful data when testing kaolinite clay samples.

TABLE 3
Kaolinite Clay (28.7% water) with 2 Minute Extraction Time

| | Mass of clay | Results (mg of | Water |
|---|---|---|---|
| Trial # | (g) | water) | in clay, wt. % |
| 1 | 5.0215 | 673 | 13.40 |
| 2 | 5.5821 | 561 | 10.05 |
| 3 | 5.182 | 907 | 17.50 |

### Example 3

In order to determine if an increased extraction time would improve results, samples from the same batch of clay as in Example 2 were tested using a fifteen minute extraction time with periodic shaking every 15 seconds. This extraction procedure was followed by a settling procedure that included centrifuging for 15 minutes. As the results in Table 4, below, indicate, the results improved over those obtained in Example 2. However, extraction efficiencies were still poor and varied significantly. Thus, an extended extraction procedure did not render the method useful on the samples of kaolinite clay.

TABLE 4
Kaolinite Clay (28.7% water) with 15 Minute Extraction Time

| | Mass of Clay | Result (mg | |
|---|---|---|---|
| Trial # | (g) | Water) | % Water in Clay |
| 1 | 5.082 | 913 | 17.97 |
| 2 | 5.0316 | 1104 | 21.94 |
| 3 | 5.0269 | 971 | 19.32 |
| 4 | 5.3318 | 1077 | 20.20 |

### Example 4

In order to even more aggressively extract water from the kaolinite clay than was done in Example 3, another experiment was run in which clay samples from the same batch as those used in Examples 2 and 3 were extracted in a mechanical tumbler for 10 minutes. This was followed by the same centrifuging procedure described in Example 3. As shown below in Table 5 the percent water calculations were more consistent with less variation using this technique, but extraction efficiencies were still low, and even with this aggressive extraction procedure, the method could not provide meaningful data.

TABLE 5
Kaolinite Clay (28.7% water) with 10 Minute Mechanical Extraction

| Trial | Mass of Clay | Result (mg | |
|---|---|---|---|
| # | (g) | Water) | % Water in Clay |
| 1 | 5.1425 | 1088 | 21.16 |
| 2 | 5.0105 | 1065 | 21.26 |
| 3 | 5.3813 | 1129 | 20.98 |
| 4 | 5.514 | 1138 | 20.64 |

### Example 5

In order to confirm that the clay samples used in Examples 2-4 had been produced properly and that the homogenization procedure had been successful, 50 gram samples of clay were placed in a drying oven at a temperature of 110° C. for 48 hours and water content was gravimetrically determined. As shown below in Table 6, the results were consistent and very close to the target preparation water percentage of 28.7%. This indicates that the batch of clay was made properly and that the sample was properly homogenized to supply consistent samples for testing.

TABLE 6
Kaolinite Clay Results with Oven Drying

| Trial | Mass Wet | Mass Dry Clay | |
|---|---|---|---|
| # | Clay(g) | (g) | % Water in Clay |
| 1 | 50.4 | 36.6 | 27.4 |
| 2 | 50.1 | 36.1 | 27.9 |
| 3 | 50.3 | 36.2 | 28 |

These results indicate that substances that can tightly bind water may be difficult to analyze using the method described in Examples 2-4. Thus, when acceptable results were achieved with cement samples, as shown below, the data were particularly surprising given that the compounds contained in a cement sample may not only adsorb water as does the clay, but can react with water to isolate it from external influences.

In the following set of experiments (Examples 6-9 below), a series of plastic concrete samples were analyzed to obtain data on precision and accuracy at water concentrations commonly encountered in the construction industry. Samples were prepared at 0, 2, 4, 6, 10, 12, 15 and 16 percent water. A ten gram concrete sample was used for each test, and three replicate samples of extract solvent were analyzed to determine repeatability within a given sample of extract. The sampling, extraction and analysis procedures were carried out as in Example 1, above. To assure that each 10 gram sample was representative, the samples were screened through a 2.36 mm mesh sieve to remove aggregate that could statistically affect results. As used herein, a “representative sample” is a sample taken from a larger batch of cement or concrete product and, when analyzed, provides results that accurately reflect the average properties of the entire batch so that an operator can obtain useful information about the batch from testing the sample. Blank samples (dry concrete containing no water) were analyzed in the same way as Example 1 to determine any response provided by the test method on samples that theoretically should provide a result of zero. An average of the resulting blank values was calculated and then subtracted from later results to compensate for any blank effect.

### Example 6

Table 7 provides results obtained from the blank analysis. Three dry, 10 gram, concrete samples (B1, B2 and B3) were sieved and extracted using dry ethanol in the same manner as Example 1, above. Three different samples of extract (0.25 mL) were removed after settling and analyzed separately as Trial 1, Trial 2 and Trial 3. Results are reported in percent water. An average result for each sample was provided and an average of each of the three trials for each of three samples (0.32%) was calculated and used as the blank value for the spiked samples that were tested later.

TABLE 7
Blank Analysis

| | | | Results in mg H₂0 | | | Results in % H₂0 | | | |
|---|---|---|---|---|---|---|---|---|---|
| | mg H₂0 added | Actual | Trial | Trial | Trial | Trial | Trial | Trial | Average |
| Sample | to sample | % H₂0 | 1 | 2 | 3 | 1 | 2 | 3 | Result |
| B1 | 0 | 0 | 28 | 32 | 56 | 0.28 | 0.32 | 0.56 | 0.39 |
| B2 | 0 | 0 | 32 | 31 | 27 | 0.32 | 0.31 | 0.27 | 0.30 |
| B3 | 0 | 0 | 31 | 34 | 20 | 0.31 | 0.34 | 0.2 | 0.28 |
| Average | | | | | | | | | 0.32 |

### Example 7

Table 8 provides results obtained from 5 different sieved concrete samples, each spiked to contain approximately 10% water. Actual water content values are provided, as well as results obtained using the procedure outlined in Example 1, above. Each sample was extracted with 100% ethanol and three different extract duplicates were analyzed from each extraction. The three trials for each sample were averaged and the blank value obtained from Table 7 was subtracted from the average. Results were recorded (Ave. Result less Blank) and a percent bias for each concrete sample was calculated. The results show accurate, precise readings for samples containing about 10% water and indicate a positive bias as analyzed. This bias is due to a 16% bias factor that is programmed into the meter to compensate for less than 100% extraction/reaction efficiencies realized when testing soil samples.

TABLE 8
Results for Concrete at 10% Water

| | | | | | | | | | Ave. | |
|---|---|---|---|---|---|---|---|---|---|---|
| | | Actual | Results in mg H₂0 | | | Results in % H₂0 | | | Result | |
| Sample | mg H₂0 | % H₂0 | Trial 1 | Trial 2 | Trial 3 | Trial 1 | Trial 2 | Trial 3 | less Blank | % Bias |
| S1 | 0.98 | 9.8 | 1111 | 1109 | 1119 | 11.11 | 11.09 | 11.19 | 11.13 | 13.57 |
| S2 | 0.97 | 9.7 | 1100 | 1107 | 1121 | 11 | 11.07 | 11.21 | 11.09 | 14.36 |
| S3 | 0.98 | 9.8 | 1117 | 1130 | 1123 | 11.17 | 11.3 | 11.23 | 11.23 | 14.62 |
| S4 | 0.98 | 9.8 | 1082 | 1093 | 1111 | 10.82 | 10.93 | 11.11 | 10.95 | 11.76 |
| S5 | 0.98 | 9.8 | 1108 | 1109 | 1099 | 11.08 | 11.09 | 10.99 | 11.05 | 12.78 |

### Example 8

Table 9 provides results obtained from 5 different sieved concrete samples each spiked to contain approximately 15% water. Actual water content values are provided as well as results obtained using the procedure outlined in Example 1 above. Each sample was extracted with 100% ethanol and three different extract duplicates were analyzed from each extraction. The three trials for each sample were averaged and the blank value obtained from Table 7 was subtracted from the average. Results were recorded (Ave. Result less Blank) and a percent bias for each soil sample was calculated. The results show accurate, precise readings for samples containing about 15% water and indicate a positive bias as analyzed. This bias is due to a 16% bias factor that is programmed into the meter to compensate for less than 100% extraction/reaction efficiencies realized when testing soil samples.

TABLE 9
Results for Concrete at 15% Water

| | | | | | | | | | Average | |
|---|---|---|---|---|---|---|---|---|---|---|
| | | Actual | Results in mg H₂0 | | | Results in % H₂0 | | | % water | |
| Sample | mg H₂0 | % H₂0 | Trial 1 | Trial 2 | Trial 3 | Trial 1 | Trial 2 | Trial 3 | less Blank | % Bias |
| S15-1 | 1.51 | 15.1 | 1596 | 1638 | 1605 | 15.96 | 16.38 | 16.05 | 16.13 | 6.82 |
| S15-2 | 1.52 | 15.2 | 1641 | 1674 | 1664 | 16.41 | 16.74 | 16.64 | 16.60 | 9.19 |
| S15-3 | 1.5 | 15 | 1631 | 1640 | 1645 | 16.31 | 16.4 | 16.45 | 16.39 | 9.24 |
| S15-4 | 1.5 | 15 | 1608 | 1665 | 1696 | 16.08 | 16.65 | 16.96 | 16.56 | 10.42 |
| S15-5 | 1.53 | 15.3 | 1647 | 1679 | 1618 | 16.47 | 16.79 | 16.18 | 16.48 | 7.71 |

### Example 9

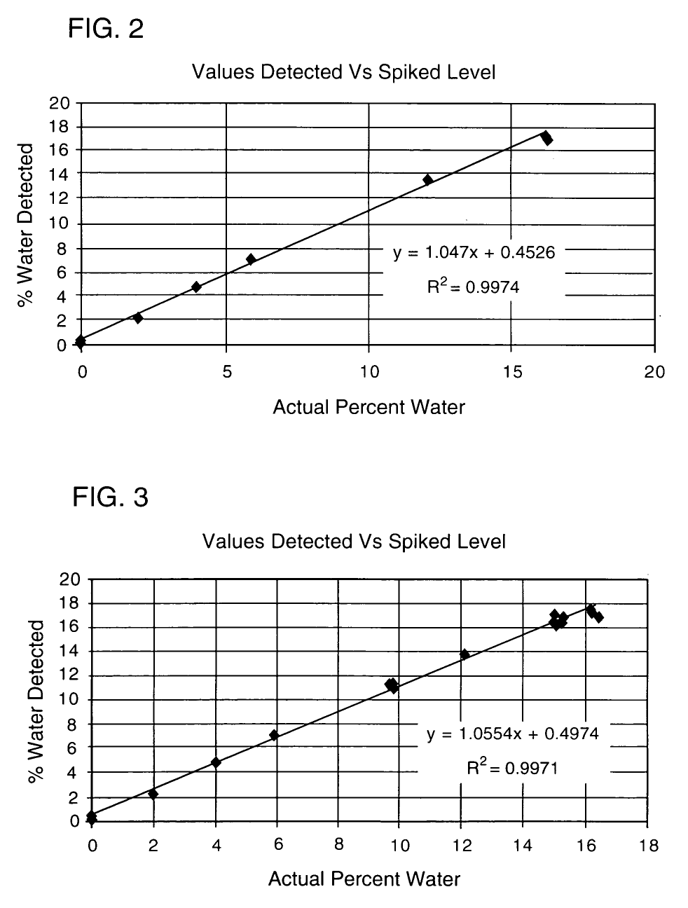

Table 10 provides results obtained from a series of concrete samples containing from 2 to 16 percent water. Two sieved (2.36 mm mesh) concrete samples at each level (2, 4, 6, 12 and 16% water) were extracted and analyzed. Each extract was sampled, reacted and analyzed three times, as above. Results from each of the three trials from each concrete sample were averaged, a blank was subtracted, and a percent bias was determined. Results indicated a greater positive bias for samples containing lesser amounts of water. Values obtained in this experiment were plotted against the actual values and a regression analysis was run. As shown in FIG. 2, a correlation coefficient of 0.9974 was realized. A 16% correction factor had been programmed into the meter to reflect extraction/reaction efficiencies that had been obtained with other sample types. A slope value slightly greater than 1 indicated a response from the test method that was slightly greater than that which had been programmed into the meter (a 16% correction factor). By correcting this slope, for example, through software or manually, more accurate results can be obtained and the bias can be compensated for. These data indicate that a correction factor of about 10%, rather than 16%, would be appropriate for cement/concrete analysis.

TABLE 10
Analysis of Concrete Samples 2-16% Water

| | | | | | | | | | Average | |
|---|---|---|---|---|---|---|---|---|---|---|
| | | Actual | Results in mg H₂0 | | | Results in % H₂0 | | | % | |
| Sample | mg H₂0 | % H₂0 | Trial 1 | Trial 2 | Trial 3 | Trial 1 | Trial 2 | Trial 3 | less Blank | % Bias |
| C2 | 0.2 | 2 | NA | 237 | 232 | NA | 2.37 | 2.32 | 2.35 | 17.25 |
| C2D | 0.2 | 2 | 219 | 225 | 220 | 2.19 | 2.25 | 2.2 | 2.21 | 10.67 |
| C4 | 0.4 | 4 | 462 | 482 | 486 | 4.62 | 4.82 | 4.86 | 4.77 | 19.17 |
| C4D | 0.4 | 4 | 473 | 482 | 462 | 4.73 | 4.82 | 4.62 | 4.72 | 18.08 |
| C6 | 0.59 | 5.9 | 709 | 708 | 706 | 7.09 | 7.08 | 7.06 | 7.08 | 19.94 |
| C6D | 0.59 | 5.9 | 695 | 686 | 691 | 6.95 | 6.86 | 6.91 | 6.91 | 17.06 |
| C12 | 1.21 | 12.1 | 1335 | 1344 | 1336 | 13.35 | 13.44 | 13.36 | 13.38 | 10.61 |
| C12D | 1.21 | 12.1 | 1365 | 1363 | 1339 | 13.65 | 13.63 | 13.39 | 13.56 | 12.04 |
| C16 | 1.62 | 16.2 | 1729 | 1705 | 1735 | 17.29 | 17.05 | 17.35 | 17.23 | 6.36 |
| C16D | 1.63 | 16.3 | 1683 | 1711 | 1684 | 16.83 | 17.11 | 16.84 | 16.93 | 3.84 |

FIG. 3 provides a regression analysis using all of the test results provided in Examples 7, 8 and 9 above. A correlation coefficient of 0.9971 was realized and, similar to the analysis in FIG. 2, a slope greater than 1 is evident.

These data indicate that the method of the present invention provides accurate, precise and repeatable results for plastic concrete samples. Accuracy may be increased by adjustment of the excessive correction factor that had been programmed into the meter. More significantly, while it was expected that results from concrete samples would be low because of the ongoing chemical reaction that immobilizes water, results indicate a complete extraction of water from the samples using a simple two minute extraction procedure. Furthermore, the combined extraction and reaction efficiency of the method appears to be greater than, or about equal to, 90%. These results are in contrast to those obtained for kaolinite clay, which under the same extraction and reaction conditions provided results that indicated actual recoveries of less than approximately 42% of the water present.

Those skilled in the art would readily appreciate that all parameters and configurations described herein are meant to be exemplary and that actual parameters and configurations will depend upon the specific application for which the systems and methods of the present invention are used. Those skilled in the art will recognize, or be able to ascertain using no more than routine experimentation, many equivalents to the specific embodiments of the invention described herein. For example, those skilled in the art may recognize that the system, and components thereof, according to the present invention may further comprise a network of systems or be a component of a system. It is, therefore, to be understood that the foregoing embodiments are presented by way of example only and that, within the scope of the appended claims and equivalents thereto, the invention may be practiced otherwise than as specifically described. The present invention is directed to each individual feature, system, or method described herein. In addition, any combination of two or more such features, systems or methods, if such features, systems or methods are not mutually inconsistent, is included within the scope of the present invention.

What is claimed is:

1. A method of determining water content in cement comprising:
providing a sample of plastic cement;
mixing a solvent with the sample to produce an extract; and
adding a reagent to the extract to produce an indicator.

2. The method of claim 1 wherein the indicator is a gas.

3. The method of claim 1 wherein the reagent is selected from calcium carbide, calcium hydride and sodium.

4. The method of claim 3 wherein the reagent is calcium hydride.

5. The method of claim 1 further comprising extracting at least 80% of the water in the plastic cement sample into the solvent.

6. The method of claim 1 further comprising adding the reagent to the solvent in a sealed compartment.

7. The method of claim 6 wherein at least a portion of the compartment is flexible.

8. The method of claim 7 wherein the maximum volume of the compartment is fixed.

9. The method of claim 2 wherein a gas is produced and the amount of gas produced is determined by measuring the pressure in the compartment.

10. The method of claim 9 wherein the pressure is measured externally of the compartment.

11. The method of claim 9 wherein the volume of the compartment is less than 100× the volume of the amount of extract to which the reagent is added.

12. The method of claim 6 wherein the compartment comprises a septum.

13. The method of claim 12 further comprising piercing the septum with a probe.

14. The method of claim 13 wherein the probe is in fluid communication with a pressure sensor.

15. The method of claim 14 wherein the pressure sensor is a pressure transducer.

16. The method of claim 15 wherein the pressure transducer provides a signal to a microprocessor.

17. The method of claim 16 wherein the microprocessor converts the signal to an amount of water.

18. The method of claim 17 further comprising the step of inputting a sample size into the microprocessor.

19. The method of claim 1 wherein the cement sample is a representative sample.

20. The method of claim 19 further comprising removing aggregate from a concrete sample to produce a representative cement sample.

21. A method for assessing the quality of concrete comprising:
extracting water from a plastic concrete sample into a solvent;
adding a reagent to the solvent;
producing a gas in an amount proportional to the amount of water in the solvent;
determining the amount of gas produced; and
assessing the quality of the concrete sample.

22. The method of claim 21 wherein the plastic concrete sample is 50 g or less.

23. The method of claim 21 wherein the reagent is added to 1 mL of solvent or less.

24. The method of claim 23 wherein the reagent is added to about 0.25 mL or less of solvent.

25. The method of claim 21 further comprising producing the gas in a sealed compartment and detecting a pressure inside the sealed compartment.

26. The method of claim 25 wherein the amount of gas produced is determined by the difference in the pressure before the reagent is added to the solvent and after the reagent has been added to the solvent.

27. The method of claim 26 wherein the pressure before the reagent is added to the solvent is atmospheric pressure.

28. The method of claim 26 further comprising determining the amount of water in the solvent by applying the ideal gas equation to the determined difference in pressure.

29. The method of claim 21 wherein the reagent is added to the solvent in a flexible container.

30. A method comprising:
extracting water from a plastic cement sample;
reacting the water with a reagent to produce a gas; and
determining the amount of gas produced.

31. The method of claim 30 wherein the amount of gas produced is determined by measuring the pressure of the gas.

32. The method of claim 31 wherein the cement sample is a representative sample.

33. The method of claim 32 further comprising removing aggregate from a concrete sample to produce a representative cement sample.
